(19) United States
(12) Patent Application Publication
(10) Pub. No.: US 2023/0372084 A1
(43) Pub. Date: Nov. 23, 2023

(54) Active Edge Protection for Balloon Expandable Valve

(71) Applicant: St. Jude Medical, Cardiology Division, Inc., St. Paul, MN (US)

(72) Inventors: Tracee Eidenschink, Wayzata, MN (US); Peter J. Ness, Minneapolis, MN (US); Tyler Govek, Minneapolis, MN (US); Michael Shane Morrissey, St. Paul, MN (US); Neil Theisen, Champlin, MN (US); Jay Reimer, Shoreview, MN (US); Preston James Huddleston, Maplewood, MN (US)

(73) Assignee: St. Jude Medical, Cardiology Division, Inc., St. Paul, MN (US)

(21) Appl. No.: 18/308,825

(22) Filed: Apr. 28, 2023

Related U.S. Application Data
(60) Provisional application No. 63/343,483, filed on May 18, 2022.

(51) Int. Cl. A61F 2/24

(52) U.S. Cl. CPC A61F 2/24; A61F 2/2418

(57) ABSTRACT

A prosthetic heart valve system may include a balloon expandable prosthetic heart valve and a delivery catheter. The delivery catheter may have a balloon on a distal end portion of the delivery catheter. In a delivery condition of the system and in an activated condition of the system, the prosthetic heart valve is mounted over the balloon so that a leading edge of the prosthetic heart valve points toward a distal end of the delivery catheter and so that the prosthetic heart valve has an outer radial extent. The delivery catheter may include a protective member operably coupled to the delivery catheter. In the delivery condition of the system, the protective member does not extend radially beyond the outer radial extent of the prosthetic heart valve. In the activated condition of the system, the protective member does extend radially beyond the outer radial extent of the prosthetic heart valve.

### CROSS-REFERENCE TO RELATED APPLICATIONS

This application claims priority to the filing date of U.S. Provisional Patent Application No. 63/343,483, filed May 18, 2022, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement (“TAVR”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to herein as a valve assembly) mounted to/within an expandable stent. In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts—including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

When self-expandable prosthetic heart valves are delivered into a patient to replace a malfunctioning native heart valve, the self-expandable prosthetic heart valve is almost always maintained in the collapsed condition within a capsule of the delivery device. While the capsule may ensure that the prosthetic heart valve does not self-expand prematurely, the overlying capsule (with or without the help of additional internal retaining features) helps ensure that the prosthetic heart valve does not come into contact with any tissue prematurely, as well as helping to make sure that the prosthetic heart valve stays in the desired position and orientation relative to the delivery device during delivery. However, balloon expandable prosthetic heart valves are typically crimped onto the balloon of a delivery device without a separate capsule that overlies and/or protects the prosthetic heart valve. One reason for this is that space is always at a premium in transcatheter prosthetic heart valve delivery devices and systems and adding a capsule in addition to the prosthetic valve and the underlying balloon may not be feasible given the size profile requirements of these procedures. Although an uncovered balloon expandable prosthetic heart valve may be at risk of moving out of the desired position relative to the delivery device during any heart valve replacement procedure, the risk may be especially acute for a transfemoral aortic valve replacement procedure as the delivery catheter needs to bend significantly when moving around the aortic arch to the native aortic valve. And while any portion of the prosthetic heart valve may be in danger of being contacted by tissue and potentially being moved to an undesired position or orientation relative to the delivery device, the leading edge of the prosthetic heart valve (which is the inflow edge in a transfemoral aortic valve replacement procedure) may be at a higher risk of such contact compared to other portions of the prosthetic heart valve.

### BRIEF SUMMARY OF THE DISCLOSURE

According to one aspect of the disclosure, a prosthetic heart valve system includes a balloon expandable prosthetic heart valve and a delivery catheter. The delivery catheter may have a balloon on a distal end portion of the delivery catheter. In a delivery condition of the system and in an activated condition of the system, the prosthetic heart valve is mounted over the balloon so that a leading edge of the prosthetic heart valve points toward a distal end of the delivery catheter and so that the prosthetic heart valve has an outer radial extent. The delivery catheter may include a protective member operably coupled to the delivery catheter. In the delivery condition of the system, the protective member does not extend radially beyond the outer radial extent of the prosthetic heart valve. In the activated condition of the system, the protective member does extend radially beyond the outer radial extent of the prosthetic heart valve.

According to another aspect of the disclosure, a prosthetic heart valve system includes a balloon expandable prosthetic heart valve and a delivery catheter. The delivery catheter may have a balloon on a distal end portion of the delivery catheter. In a delivery condition of the system and in an activated condition of the system, the prosthetic heart valve is mounted over the balloon so that a leading edge of the prosthetic heart valve points toward a distal end of the delivery catheter. The delivery catheter may include a nosecone coupled to a shaft that extends though the delivery catheter, the shaft being translatable relative to the balloon to translate the nosecone relative to the balloon. In a delivery condition of the system, the nosecone is positioned at a first axial location relative to the balloon so that a portion of the balloon and the leading edge of the prosthetic heart valve are covered by a proximal portion of the nosecone. In a deployment condition of the system, the nosecone is positioned at a second axial location relative to the balloon distal to the first axial location, so that the proximal portion of the nosecone does not overlie the leading edge of the prosthetic heart valve.

### BRIEF DESCRIPTION OF THE DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 1K illustrates the view of FIG. 1H with an additional outer cuff provided on the stent.

FIG. 2A illustrates a prosthetic heart valve crimped over a balloon of a delivery device.

FIG. 2B is a schematic view of the balloon of FIG. 2A after having been inflated.

FIG. 3 is a perspective view of a prosthetic heart valve delivery device having protective wires in a deployed condition.

FIG. 4A is a side view of a prosthetic heart valve delivery device having protective wires in a stowed condition according to another aspect of the disclosure.

FIGS. 4B-C are side and end views, respectively, of the prosthetic heart valve delivery device of FIG. 4A with the protective wires in a deployed condition.

FIGS. 5A-B are side and front views, respectively, of a protective wire according to another aspect of the disclosure.

FIG. 5C is a side view of the protective wire of FIGS. 5A-B passing through a balloon of a balloon catheter.

FIG. 5D is a highly schematic illustration of the protective wire of FIG. 5A being introduced into a vessel by an introducer.

FIG. 6A is a side view of a distal end of a balloon catheter with a protective wire in a deployed condition according to an aspect of the disclosure.

FIG. 6B is a side view of the distal end of the balloon catheter and protective wire of FIG. 6A positioned within the aortic arch.

FIG. 7A is a side view of the distal end of a protective wire in an inflated condition.

FIG. 7B is a side view of the distal end of a protective wire positioned through a balloon catheter while in an inflated condition.

FIG. 7C is a side view of an alternate embodiment of an inflatable bumper generally similar in function to that of FIGS. 7A-B.

FIGS. 8A-B are schematic views of a balloon catheter with a protective member, according to another aspect of the disclosure, in a stowed and deployed condition, respectively.

FIG. 8C is highly schematic view of an alternate version of the balloon catheter of FIGS. 8A-B with a protective member in a deployed condition.

FIGS. 8D-E are schematic views of an alternate version of the balloon catheter of FIGS. 8-B, with a protective member in a stowed and deployed condition, respectively

FIGS. 9A-B are schematic views of a balloon catheter with a protective member, according to a further aspect of the disclosure, in a stowed and deployed condition, respectively.

FIG. 9C is a schematic view of an alternate version of the balloon catheter of FIGS. 9A-B.

FIGS. 10A-C are schematic views of another version of a balloon catheter with a protective member in different stages of deployment of the protective member.

FIGS. 11A-B are schematic views of a balloon catheter with a protective member, according to a different aspect of the disclosure, in a stowed and deployed condition, respectively.

FIG. 11C is a cross-section of the protective member of FIG. 11A taken along the section line 11C-11C of FIG. 11A.

FIG. 11D is a cross-section of the protective member of FIG. 11B taken along the section line 11D-11D of FIG. 11B.

### DETAILED DESCRIPTION

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to a trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

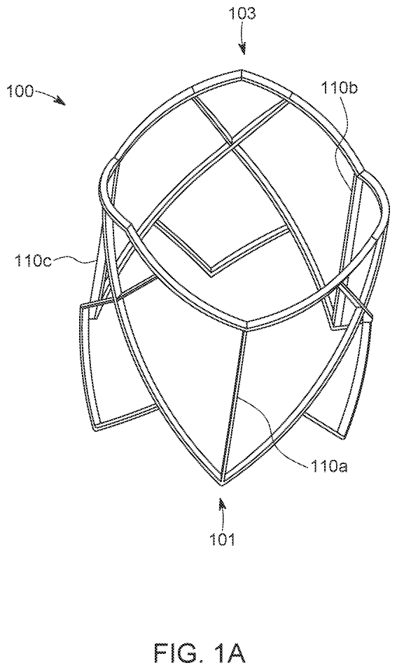

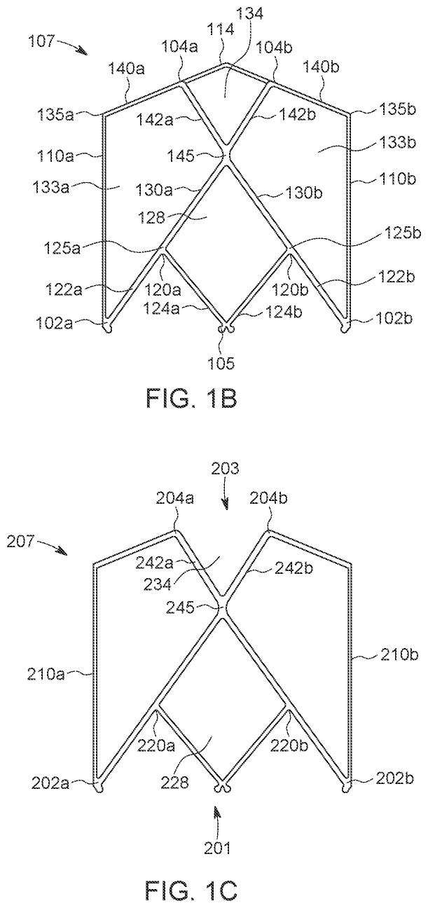

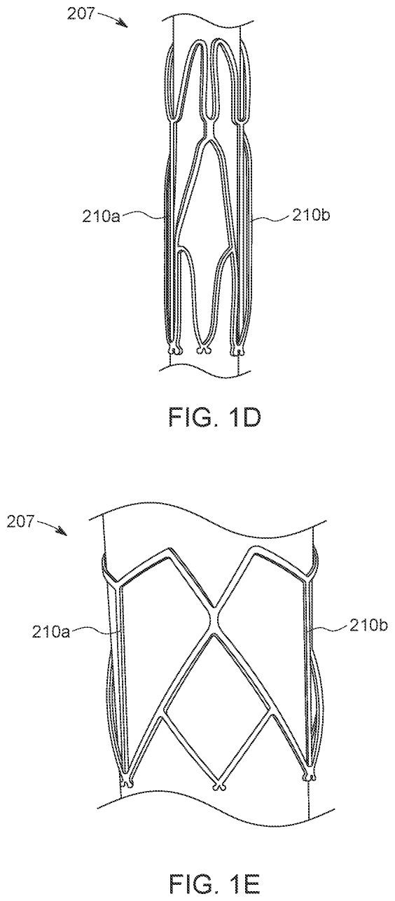

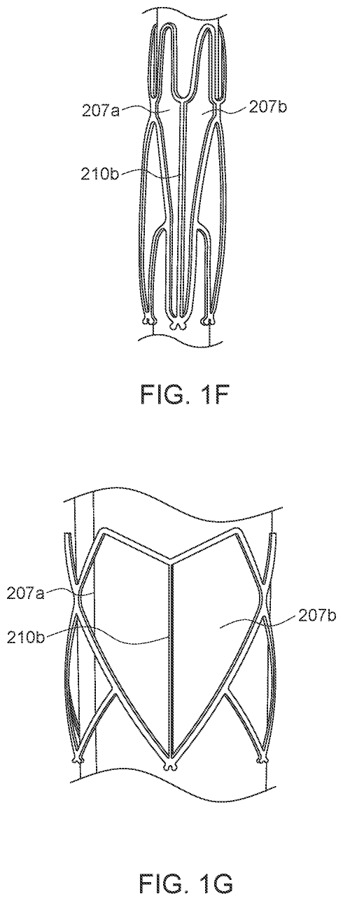

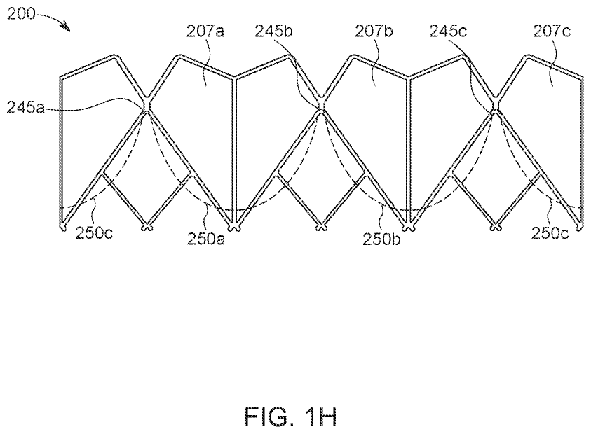

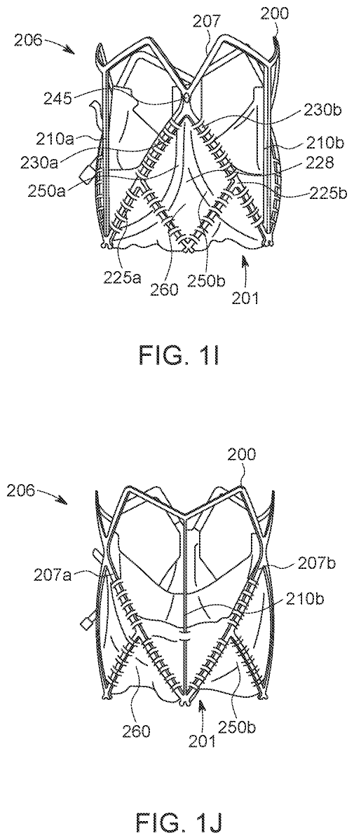

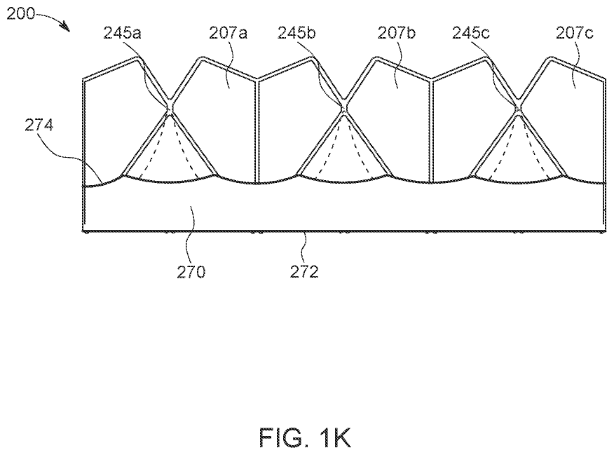

FIG. 1A illustrates a perspective view of a stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100 and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail, and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135 and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 260 may include an inflow end disposed substantially along inflow end 201 of stent 200. FIG. 1I shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular leakage or “PV” leakage). In the embodiment illustrated in FIGS. 1I-J, the cuff 260 only covers about half of the stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

As noted above, FIGS. 1I-J illustrate a cuff 260 positioned on an interior of the stent 200. An example of an additional outer cuff 270 is illustrated in FIG. 1K. It should be understood that outer cuff 270 may take other shapes than that shown in FIG. 1K. The outer cuff 270 shown in FIG. 1K may be included without an inner cuff 260, but preferably is provided in addition to an inner cuff 260. The outer cuff 270 may be formed integrally with the inner cuff 260 and folded over (e.g. wrapped around) the inflow edge of the stent, or may be provided as a member that is separate from inner cuff 260. Outer cuff 270 may be formed of any of the materials described herein in connection with inner cuff 260. In the illustrated embodiment, outer cuff 270 includes an inflow edge 272 and an outflow edge 274. If the inner cuff 260 and outer cuff 270 are formed separately, the inflow edge 272 may be coupled to an inflow end of the stent 200 and/or an inflow edge of the inner cuff 260, for example via suturing, ultrasonic welding, or any other suitable attachment modality. The coupling between the inflow edge 272 of the outer cuff 270 and the stent 200 and/or inner cuff 260 preferably results in a seal between the inner cuff 260 and outer cuff 270 at the inflow end of the prosthetic heart valve so that any retrograde blood that flows into the space between the inner cuff 260 and outer cuff 270 is unable to pass beyond the inflow edges of the inner cuff 260 and outer cuff 270. The outflow edge 274 may be coupled at selected locations around the circumference of the stent 200 to struts of the stent 200 and/or to the inner cuff 260, for example via sutures. With this configuration, an opening may be formed between the inner cuff 260 and outer cuff 270 circumferentially between adjacent connection points, so that retrograde blood flow will tend to flow into the space between the inner cuff 260 and outer cuff 270 via the openings, without being able to continue passing beyond the inflow edges of the cuffs. As blood flows into the space between the inner cuff 260 and outer cuff 270, the outer cuff 270 may billow outwardly, creating even better sealing between the outer cuff 270 and the native valve annulus against which the outer cuff 270 presses. The outer cuff 270 may be provided as a continuous cylindrical member, or a strip that is wrapped around the outer circumference of the stent 200, with side edges, which may be parallel or non-parallel to a center longitudinal axis of the prosthetic heart valve, attached to each other so that the outer cuff 270 wraps around the entire circumference of the stent 200.

The stent may be formed from biocompatible materials, including metals and metal alloys such as cobalt chrome (or cobalt chromium) or stainless steel, although in some embodiments the stent may be formed of a shape memory material such as nitinol or the like. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example including transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstance, under an overlying sheath). Upon arrival at or adjacent the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

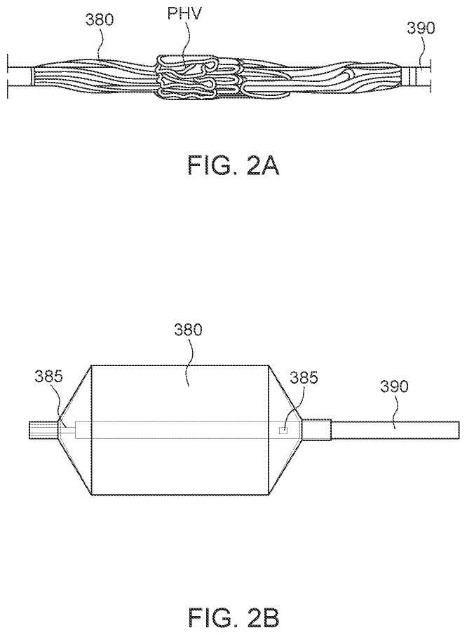

Referring to FIG. 2A, an example of a prosthetic heart valve PHV, which may include a stent similar to stents 100 or 200, is shown crimped over a balloon 380 of a balloon catheter 390 while the balloon 380 is in a deflated condition. It should be understood that other components of the delivery device, such as a handle used for steering and/or deployment, as well as a syringe for inflating the balloon 380, are omitted from FIGS. 2A-B. The prosthetic heart valve PHV may be delivered intravascularly, for example through the femoral artery, around the aortic arch, and into the native aortic valve annulus, while in the crimped condition shown in FIG. 2A. Once the desired position is obtained, fluid may be pushed through the balloon catheter 390 to inflate the balloon 380, as shown in FIG. 2B. FIG. 2B omits the prosthetic heart valve PHV, but it should be understood that, as the balloon 380 inflates, it forces the prosthetic heart valve PHV to expand into the native aortic valve annulus (although it should be understood that other heart valves may be replaced using the concepts described herein). In the illustrated example, fluid flows from a syringe (not shown) into the balloon 380 through a lumen within balloon catheter 390 and into one or more ports 385 located internal to the balloon 380. In the particular illustrated example of FIG. 2B, a first port 385 may be one or more apertures in a side wall of the balloon catheter 390, and a second port 385 may be the distal open end of the balloon catheter 390, which may terminate within the interior space of the balloon 380.

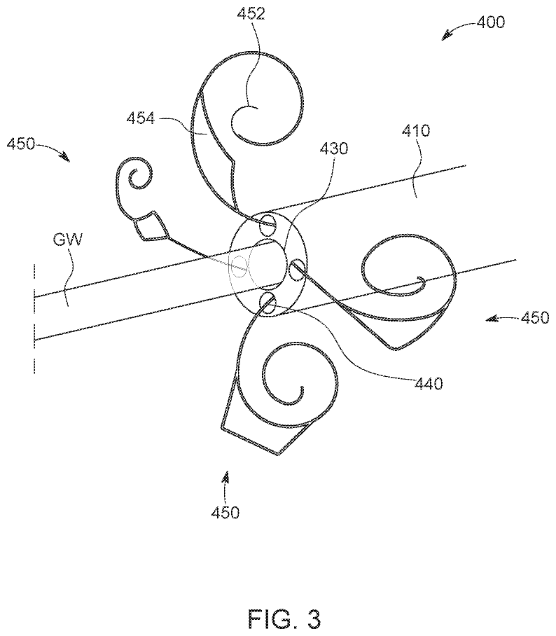

FIG. 3 illustrates a distal end of a delivery device 400 for delivering and deploying a balloon expandable prosthetic heart valve PHV. The prosthetic heart valve PHV used with delivery device 400 may be one of the balloon expandable prosthetic heart valves described above, or indeed any balloon expandable prosthetic heart valve. It should be understood that various components of delivery device 400 are omitted for clarity, for example a control handle at or near the proximal end of the delivery device, the balloon that is to be inflated to deploy the prosthetic heart valve PHV, as well as the prosthetic heart valve PHV itself. In particular, FIG. 3 illustrates a distal end portion of a delivery device 400, including a balloon catheter 410 which includes a balloon (not pictured) over which the prosthetic heart valve PHV (also not pictured) would be crimped during delivery. The balloon catheter 410 may include a central lumen 430 extending therethrough, which may be a guidewire lumen so that the balloon catheter 410 may be advanced over a guidewire GW that has previously been delivered to or near the native heart valve annulus to be replaced. In addition to the central lumen 430, a plurality of peripheral lumens 440. In the illustrated embodiment, four peripheral lumens 440 are shown, each positioned radially outward of the central lumen 430 and being positioned about 90 degrees apart from each other. As few as two peripheral lumens 440 may instead be provided, or as many as eight or more peripheral lumens 440 may be provided, and although the peripheral lumens are preferably evenly spaced, they may have any desirable spacing relative to each other.

Each peripheral lumen 440 may terminate at the distal end of the balloon catheter 410, just distal of the balloon and the prosthetic heart valve PHV configured to be crimped over the balloon. The proximal ends of the peripheral lumens 440 may be open at a proximal end of the delivery device, or may terminate at or near the handle so that the protective wires 450, described in greater detail below, are controllable via one or more actuators on the handle. A protective wire 450 may extend through each peripheral lumen 440. In the illustrated example, the protective wires 450 may be formed of a shape-memory material such as a nickel titanium alloy like nitinol, and each protective wire 450 includes a distal free end 452. The proximal end (not shown) of each protective wire 450 may be free and extend through the proximal end of the delivery device 400 so that a user may grasp and manipulate the protective wires 450 (e.g. by pulling or pushing the protective wires 450 through the peripheral lumens 440). In another embodiment, the proximal ends of each protective wire 450 may be coupled to an actuator on the handle of the delivery device 400, so that sliding the actuator translates the protective wires 450 through the peripheral lumens 440.

The distal ends of the protective wires 450 may be shape-set, for example via heat setting, so that the distal ends tend to curl away from the central lumen 430 (e.g. curl back toward the proximal end of the delivery device 400). Stated in another way, the distal ends of the protective wires 450 may be shape-set so that the distal-facing portions of the wires extending just beyond the peripheral lumens 440 have a generally convex curvature while the opposite surfaces have a generally concave curvature. In the absence of applied forces, the distal ends of the protective wires 450 may curl less than or, as shown, more than 360 degrees. Each protective wire may be formed as only a single wire, or other shapes and/or features may be provided. For example, as illustrated, a distal end portion of each protective wire 450 includes a generally diamond-shaped structure 454 that is capable of collapsing while inside the peripheral lumen 440 and expand to the illustrated diamond shape when extended beyond the end of the peripheral lumen 440.

In use, the prosthetic heart valve PHV would be crimped over the deflated balloon near the distal end of the balloon catheter 410, and the protective wires 450 would each be fully withdrawn into their respective peripheral lumens 440 prior to introducing the balloon catheter 410 into the patient. The central lumen 430 of the balloon catheter 410 may be threaded over the guidewire GW and the balloon catheter 410 may be advanced into the patient. When the distal end of the balloon catheter 410 reaches near the beginning of the aortic arch, the protective wires 450 may be advanced distally. As the protective wire 450 exit their respective peripheral lumens 440, they begin to take their set shape and curl proximally, as shown in FIG. 3. In this condition, the distal ends of the protective wires 450 extend radially outwardly a distance greater from the outer surface of the balloon catheter 410 than the distance which the prosthetic heart valve PHV extends from the outer surface of the balloon catheter 410. With this configuration, the leading edge of the crimped prosthetic heart valve will be protected by the protective wires 450. In other words, any tissue that would be at risk of coming into contact with the leading edge of the prosthetic heart valve during delivery will instead contact the protective wires 450, thus shielding the leading edge of the prosthetic heart valve from being contacted and potentially shifted out of the desired position and orientation relative to the collapsed balloon. In the illustrated embodiment, the diamond 454 may provide increased protective area compared to if the protective wires 450 were formed only as a single wire without the diamond-shape feature. By forming the leading-most end of the protective wires 450 to present a convex contour, as the delivery device 400 advances distally through the aortic arch, the protective wires 450 may contact the Although the protective wires 450 are described above as being activated or deployed only when the balloon catheter 410 nears the aortic arch, it should be understood that the protective wires 450 may be deployed earlier in the procedure if desired, for example including during insertion (whether sheathless or through a sheath, such as an expandable sheath). Thus, for all embodiments described herein, the active edge protection may have utility during traversing the aortic arch, during insertion into the patient, or at any other desired point during the procedure. And it should be understood that the protective wires 450 may be used in delivery systems 400 using other deliver routes besides transfemoral delivery. In other words, the protective wires 450 may be useful in anatomy other than only at the aortic arch. Once the distal end of the balloon catheter 410 is at or near the native valve annulus (e.g., the native aortic valve), the protective wires 450 may be withdrawn back into their respective peripheral lumens 440 so that the balloon may be expanded and the prosthetic heart valve may be deployed into the native valve annulus without interference from the protective wires 450. After the implantation is complete, the protective wires 450 may remain withdrawn in their respective peripheral lumens 430 so that the protective wires 450 do not cause trauma to the patient's tissue.

It should be understood that, although not shown, the balloon catheter 410 may include additional lumens, such as inflation lumens to allow fluid (e.g., a liquid such as saline) to pass through the balloon catheter 410 to inflate the balloon (not illustrated) to deploy the prosthetic heart valve PHV.

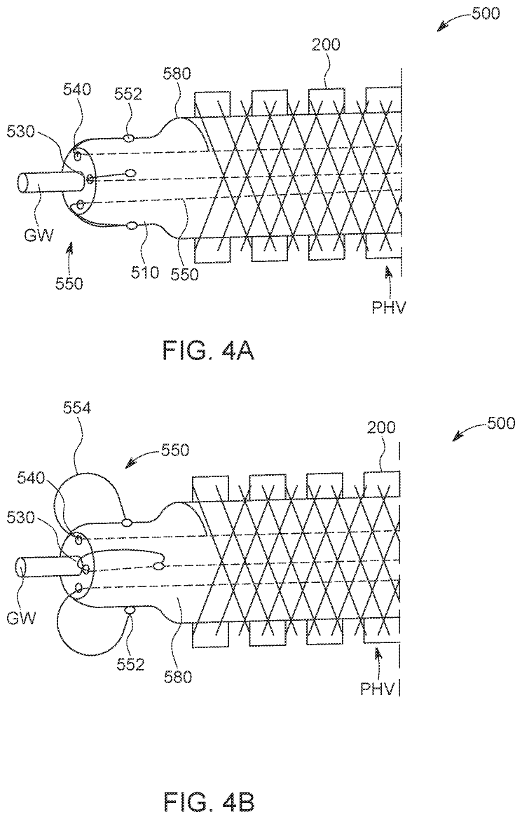

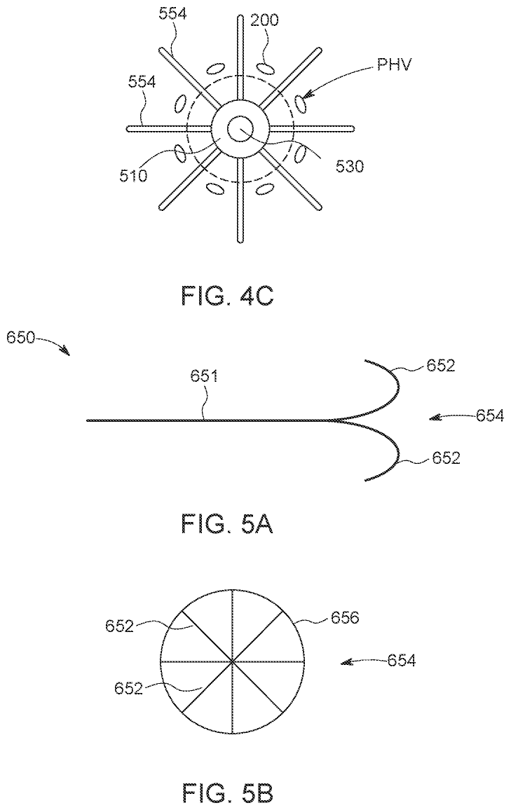

FIG. 4A illustrates a distal end of a delivery device 500 for delivering and deploying a balloon expandable prosthetic heart valve PHV. The prosthetic heart valve PHV used with delivery device 500 may be any suitable balloon expandable prosthetic heart valve, including any one described above, including one that incorporates stent 200. As with delivery device 400, certain components of delivery device 500 are omitted for clarity, for example a control handle at or near the proximal end of the delivery device. However, unlike FIG. 3, FIGS. 4A-B illustrate the prosthetic heart valve PHV in a crimped condition over a deflated balloon 580 of the balloon catheter 510. The balloon catheter 510 may include a central lumen 530 extending therethrough for accepting a guidewire GW similar to delivery device 400. Also, as with delivery device 400, balloon catheter 510 may include peripheral lumens 540 in the same way as described in connection with peripheral lumens 440 (including the alternatives described therewith). Thus, the specific embodiment of FIGS. 4A-C includes four peripheral lumens 540 positioned radially outward of the central lumen 530, with each peripheral lumen 540 being evenly spaced from an adjacent one of the peripheral lumens 540.

In the illustrated example, the protective wires 550 extending through their respective peripheral lumens 540 may be formed of a shape-memory material such as nitinol, although protective wires 550 may be formed of other materials, including non-shape-memory materials, such as stainless steel or any suitable polymer such as Pebax. Whereas protective wires 450 each include a distal free end 452, each protective wire 550 includes a distal fixed end 552. Similar to protective wires 450, each protective wire 550 may have a proximal end that is coupled to an actuator (e.g., on the delivery device handle) or is otherwise available for manipulation by a user. The distal fixed end 552 of each protective wire 550 may be coupled to the balloon catheter 510 just distal to the balloon 580, and the coupling may be via any suitable mechanism, for example via adhesives, welding, mechanical couplings, or any other suitable mechanism.

In the stowed condition, shown in FIG. 4A, the proximal ends of the protective wires 550 may each be positioned relatively proximally so that the protective wires 550 are relatively taut. In other words, the protective wires 550 extend distally through the peripheral lumens 540 (the internal extension of protective wires 550 shown in dashed lines in FIG. 4A), and at the point that the protective wires 550 exit the distal end of the peripheral lumens 540, the protective wires 550 tightly turn back proximally to their point of connection 552 to the balloon catheter 510. Thus, in the stowed condition, no part of the protective wires 550 extend radially outwardly beyond the prosthetic heart valve PHV.

In the deployed or actuated condition of the protective wires 550, shown in FIGS. 4B-C, the distal ends of the protective wires 550 form a looped portion 554 extending from the connection point 552 to the point at which the protective wires 550 enter their respective peripheral lumens 540. The protective wires 550 may be transitioned to the deployed or actuated condition by advancing the proximal ends of the protective wires 550 distally. Because the distal-most ends of the protective wires 550 are fixed to the balloon catheter 510, the protective wires 550 tend to form a loop or curved shape upon actuation. This may occur whether or not the protective wires 550 are formed of shape-memory materials that are shape-set to take on the illustrated loop shape. When the protective wires 550 are actuated to form the looped portions 554, as best shown in FIG. 4C, the looped portions 554 extend radially outwardly beyond the radially extend of the prosthetic heart valve PHV. Delivery device 500 may be used in substantially the same fashion as delivery device 400, so an exemplary use is not described again in detail. The main difference is that the distal-most ends 552 of the protective wires 550 are always positioned outside of the peripheral lumens 540. Otherwise, the functionality and use of the delivery device 500 and protective wires 550 is substantially identical to the functionality and use of the delivery device 400 and protective wires 450.

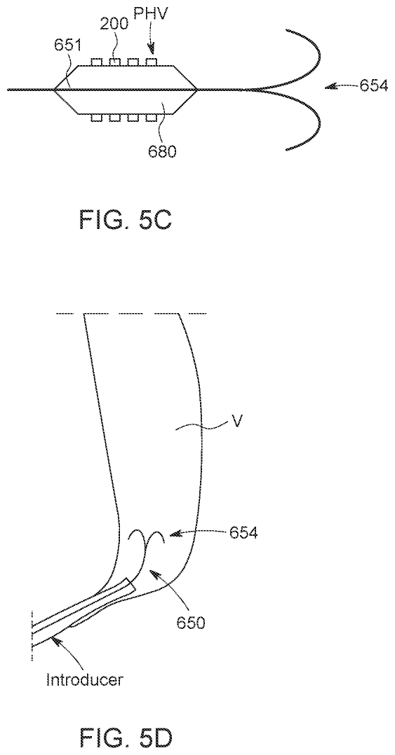

FIG. 5A is a side view of a protective wire 650 according to another embodiment of the disclosure, the protective wire 650 being shown in a deployed condition. Preferably, protective wire 650 is formed of a shape-memory material, such as nitinol, although other shape-memory materials may be suitable. As with other embodiments herein, it should be understood that the term “wire” does not necessarily require any specific material—e.g., in some embodiments, the protective wire need not be formed of a metal or metal alloy. In the illustrated embodiment, the protective wire 650 includes a main shaft portion 651 sized and shaped to extend through a lumen of a delivery device, described in greater detail below. The distal or leading end of the protective wire 650 may be shape-set (e.g., via heat setting) so that, in the absence of applied forces, the protective wire 650 forms a distal end cap 654. The end cap 654 may be formed by one or more (preferably three, four, or more) individual wire portions 652 that at first extend distally from the main shaft 651, and then extend in the proximal direction a short distance to present a generally convex contour at the leading end of the protective wire 650. Although only two such wire portions 652 are visible in the side view of FIG. 5A, more than two wire portions 652 may be included, as shown in the front or end view of FIG. 5B. In the specific embodiment shown in FIG. 5B, the main shaft 651 transitions into eight individual wire portions 652 that each hook proximally, forming a shape similar to the framework for an umbrella. In some embodiments, a material may cover and/or span across the individual wire portions 652. For example, as shown in FIG. 5B, an optional covering 656 is provided that spans across the individual wire portions 652. The combination of the covering 656 and individual wire portions 652 may be conceptually similar to the material that covers the framework of an umbrella to help protect objects positioned underneath the covering 656. The covering 656 may be formed of any suitable material, such as polyurethane. It should be understood that, the protective wire 650, including the individual wire portions 652 and any covering 656 provided therewith, may be able to transition into a substantially straight condition to pass through a generally cylindrical or circular lumen of a delivery device, with the individual wire portions 652 and any associated covering 656 being able to transition to the set shape after exiting from the constraints of such a lumen.

In use, as shown in FIG. 5C, the protective wire 650 may extend through a lumen (e.g., a center lumen or a peripheral lumen) of the balloon catheter onto which balloon 680 is mounted, with the prosthetic heart valve PHV (including stent 200) being crimped over the balloon 680 while the balloon 680 is in a deflated condition. It should be understood that, in the view of FIG. 5C, the catheter structure of the balloon catheter is omitted from the view for purpose of clarity. In use, the balloon catheter may have a structure similar to other balloon catheters described herein, or other generally known balloon catheters. The protective wire 650 may be transitioned into the deployed or activated condition shown in FIG. 5C at any point during the delivery process, including at the very beginning (e.g., upon entry into the patient) or at a later time (e.g., just before traversing the aortic arch). When the protective wire 650 is in the deployed condition shown in FIG. 5C, the protective wire may be moved proximally relative to the balloon 680 and prosthetic heart valve PHV, if desired, to be positioned as closed to the prosthetic heart valve PHV as desired, including such that portions of the cap 654 actually partially surround the prosthetic heart valve PHV. However, the cap 654 need not actually partially surround the prosthetic heart valve PHV to provide the protective effect.

The use of the protective wire 650 may otherwise be similar to the uses described above. In other words, because the cap 654, when deployed, is positioned distally to the prosthetic heart valve PHV and extends radially beyond the radial extent of the crimped prosthetic heart valve PHV, as the assembly is advanced through the vasculature, the deployed cap 654 will contact tissue that might otherwise directly contact the prosthetic heart valve PHV, particularly the leading edge of the prosthetic heart valve PHV. Thus, the position and shape of the deployed cap 654 helps to protect the prosthetic heart valve PHV from unintentionally contacting any tissue which might cause a loss of the desired position and/or orientation of the prosthetic heart valve PHV relative to the balloon 680 and/or the balloon catheter. When the prosthetic heart valve PHV is at or near the final deployment site (e.g., the native aortic valve annulus), the main shaft 651 of the protective wire 650 may be withdrawn proximally, forcing the cap 654 to generally flatten as it is pulled within the lumen through which the protective wire 650 extends. Then, the balloon 680 may be inflated to deploy the prosthetic heart valve PHV without interference from the protective wire 650, and the protective wire 650 has been either fully withdrawn from the body or otherwise stowed away within a lumen of the interior of the balloon catheter. As should be understood from the description above, the cap 654 may function suitably with or without the additional covering 656, but the additional covering 656 may provide additional protection if used.

FIG. 5D illustrates an alternate use of the protective wire 650, although the protective mechanism is still substantially the same. Rather than the protective wire 650 passing directly through the balloon catheter onto which the prosthetic heart valve PHV is crimped, the protective wire 650 may extend through a lumen formed in a side wall of an introducer. As shown in FIG. 5D, the protective wire 650 may extend through the side wall of the introducer which has been introduced into a blood vessel V, so that the main center lumen of the introducer remains available for the balloon catheter to pass through. Otherwise, the operation is substantially similar, with the end cap 654 being positioned distally to the balloon catheter (and the prosthetic heart valve PHV received thereon) to help protect directed contact between the prosthetic heart valve PHV and the anatomy which might otherwise cause an undesirable shift in the position and/or orientation of the prosthetic heart valve PHV.

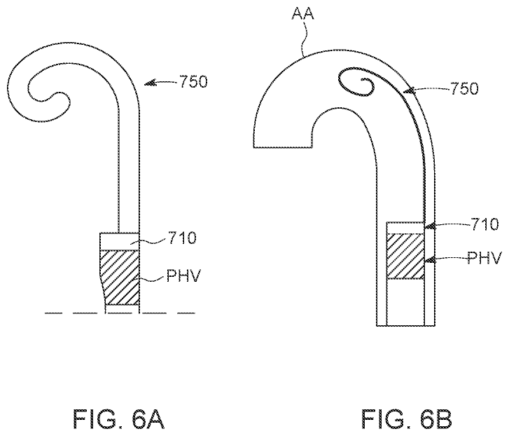

FIG. 6A illustrates a side view of a distal portion of a balloon catheter 710 with a prosthetic heart valve PHV crimped over a balloon (not separately shown) in a delivery condition. The balloon catheter 710 may be similar to those described above, or any generic balloon catheter, and may include a center lumen for riding over a guidewire (not shown). The balloon catheter 710 may also include one or more peripheral lumens similar to peripheral lumen(s) 440 balloon catheter 410. A protective wire 750 may extend through a peripheral lumen. In the illustrated embodiment, the protective wire 750 is in an actuated or deployed condition. The protective wire 750 is preferably formed from a shape-memory material, such as nitinol, and has the ability to lie straight within the peripheral lumen or take a set-shape in the absence of applied forces (e.g., after exiting the constraint of the peripheral lumen). The protective wire 750 may be shape-set (e.g., by heat setting) to have a distal tip that curves proximally backwards so as to present an atraumatic leading end. Whereas the protective wires described above generally function as a “bumper” of sorts, protective wire 750 is configured to help maintain the prosthetic heart valve PHV away from the tissue wall during delivery. For example, as shown in FIG. 6B, the balloon catheter 710 is approaching the aortic arch AA. During delivery, the protective wire 750 may be stowed in a substantially straight condition within the peripheral lumen of the balloon catheter 710. As the balloon catheter 710 approaches the aortic arch AA (or, if desired, at an earlier point), the protective wire 750 may be advanced distally so that the distal end of the protective wire 750 clears the peripheral lumen and takes its set-shape. Preferably, the peripheral lumen housing the protective wire 750 is positioned adjacent the outside bend of the aortic arch AA (e.g., the side that is positioned relatively superiorly at the superior-most aspect of the aortic arch AA, or the side with the large radius of curvature). As should be understood, as the balloon catheter 710 is advanced through the aortic arch AA toward the native aortic valve (not shown in FIG. 6B), the balloon catheter 710 will tend to “want to” track along the outer bend of the aortic arch AA. This may not be desirable, for example because there may be a relatively high likelihood of the inflow end of the prosthetic heart valve PHV contacting tissue at the outer bend of the aortic arch AA and undesirably shifting the position or orientation of the prosthetic heart valve PHV relative to the balloon and/or balloon catheter 710. Prior to advancing the balloon catheter 710 through the aortic arch AA, however, the protective wire 750 may be deployed and form the set-shape, in which the distal tip of the protective wire 750 curves proximally and away from the outer bend of the aortic arch AA. With the protective wire 750 deployed, the protective wire 750 will tend to contact the outer bend of the aortic arch AA. The force on the protective wire 750 from contact with the outer bend of the aortic arch AA will tend to move the distal tip of the balloon catheter 710 (and thus the prosthetic heart valve PHV mounted thereon) slightly away from the wall of the aortic arch AA, helping to avoid direct contact between the prosthetic heart valve PHV and the wall of the aortic arch AA as the balloon catheter 710 advances through the aortic arch AA. In some uses, the protective wire 750 may remain stationary relative to the anatomy as the balloon catheter 710 rides over the protective wire 750. In other uses, the protective wire 750 may move in conjunction with the balloon catheter 710, for example with the protective wire 750 extending beyond the balloon catheter 710 by a set distance.

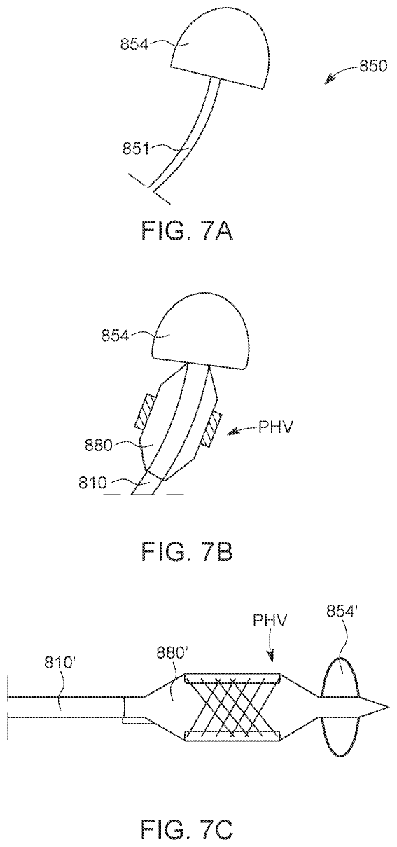

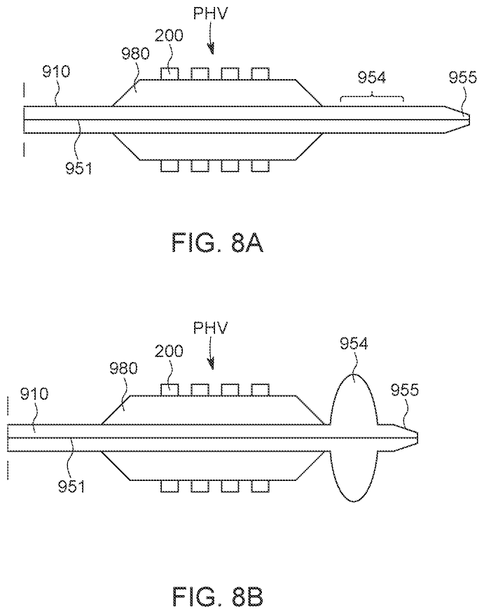

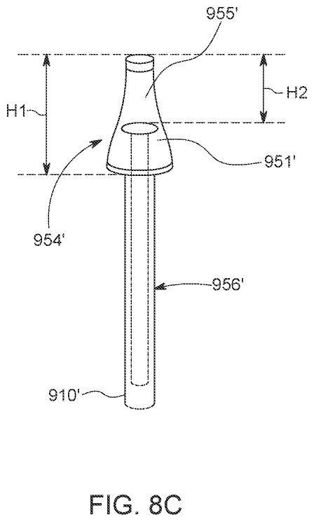

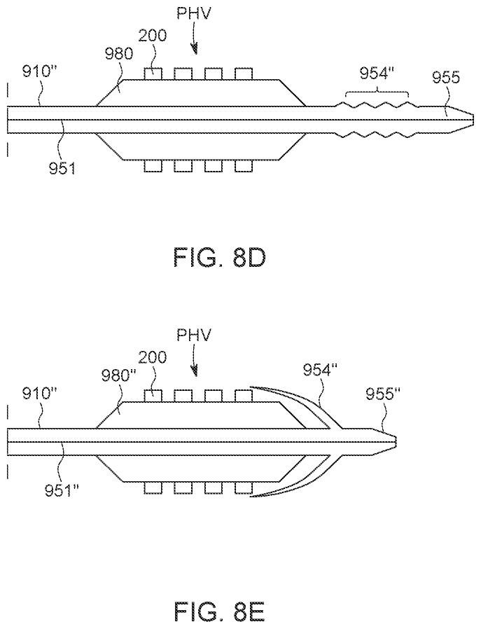

FIG. 7A is a side view of a distal portion of a protective wire 850 according to another aspect of the disclosure. As noted above, the term “wire” does not necessarily require any specific material. In the illustrated embodiment, protective wire 850 includes a main shaft 851 that terminates in an inflatable cap 854. The cap 854 is shown in FIG. 7A in the inflated condition and takes the shape of a hemisphere (or a bullet, bullnose, or cone) when inflated, although other shapes may be suitable. The main shaft 851 may be formed of any suitable material, but preferably includes an inflation lumen that fluidly connects to the interior volume of the cap 854 and is preferably strong enough to be relatively easily pushed distally through a lumen (e.g., a center or peripheral lumen) of the balloon catheter 810. The cap 854 may be formed of any suitable material, including compliant, semi-compliant, or noncompliant balloon materials, including materials similar to that forming balloon 880. In some embodiments, perfusion channels may be provided (e.g., within or on cap 854) to allow for blood to flow across or through the protective wire, even while actively protecting the prosthetic heart valve. Perfusion channels may similarly be provided in other embodiments described herein, for example in protective member 954 describe below in connection with FIGS. 8A-B.

The protective wire 850 may be partially or fully housed within a lumen of the balloon catheter 810 when the cap 854 is deflated. In other words, in the deflated condition, the cap 854 is capable of fitting fully inside a lumen of the balloon catheter 810. At any desired point during delivery of the prosthetic heart valve PHV, for example just after insertion into the patient, or just prior to moving through the aortic arch, the main shaft 851 of the protective wire 850 may be advanced until the cap 854 is free from the lumen of the balloon catheter 810. Then, the cap 854 may be inflated, for example by passing a fluid (including a liquid such as saline) through the interior lumen of the main shaft 851 and into the cap 854, causing the cap 854 to inflate into the generally hemispherical shape shown in FIG. 7B. When inflated, cap 854 may be positioned just distal to the crimped prosthetic heart valve PHV. Preferably, if the cap 854 has a hemispherical shape when inflated, the spherical portion of the shape is at the leading or distal end of the protective wire 850, with the generally flat side on the trailing end of the cap 854. Further, the diameter of the inflated cap 854 is preferably large enough so that it extends radially outward of the radially outward-most extent of the crimped prosthetic heart valve PHV. With this configuration, as the balloon catheter 810 and protective wire 850 are advanced distally through the vasculature, the prosthetic heart valve PHV will be protected, as a result of the inflated cap 854, from directly contacting tissue and being undesirable repositioned or reoriented relative to the balloon 880 and/or balloon catheter 810. When it is time to inflate the balloon 880 to deploy the prosthetic heart valve PHV, the cap 854 may first be deflated by withdrawing the fluid from the cap 854 through the lumen of the main shaft 851, and then the main shaft 851 may be retracted until the deflated cap 854 is fully positioned within a lumen of the balloon catheter 810 (or otherwise fully withdrawn from the patient). Thus, as the prosthetic heart valve PHV is expanded into the native valve annulus, the cap 854 will not interfere with such deployment.

FIG. 7C illustrates an alternate embodiment of inflatable cap 854′ on a balloon catheter 810′. Instead of being part of a separate protective wire, the inflatable cap 854′ may be directly positioned on the outer surface of the balloon catheter 810′, just distal to the balloon 880′ and the prosthetic heart valve PHV crimped thereon. The cap 854′ may be formed of urethane or another inflatable material, and an inflation lumen may run through the balloon catheter 810′ and into the interior volume of the cap 854′. When inflated, the cap 854′ may have a generally doughnut or toroidal shape, with structure of the balloon catheter 810′ passing through the center of the cap 854′. Similar to the embodiment of FIGS. 7A-B, when the cap 854′ is inflated, it extends radially beyond the outer-most radial extent of the crimped prosthetic heart valve PHV, providing substantially the same function as described in connection with the cap 854 of FIGS. 7A-B. As with cap 854, cap 854′ may be inflated just prior to moving through the aortic arch, or otherwise at any time desired during the delivery of the prosthetic heart valve PHV to the native valve annulus. Cap 854′, as well as cap 854, may additionally provide a centering functionality when inflated, tending to force the balloon catheter 810′ (or 810) toward the center of the vessel with which the inflated cap 854′ (or 854) is contacting. For the embodiment of FIG. 7C, it may not be necessary to deflate the cap 854′ just prior to deployment of the prosthetic heart valve PHV, but it nonetheless may be desirable to do so to avoid any interference between the inflated cap 854′ and the patient's anatomy or with the deployment of the prosthetic heart valve PHV.

FIGS. 8A-B illustrate schematic views of a balloon catheter 910 with a protective member 954 in a stowed and deployed condition, respectively. Balloon catheter 910 may be of generally similar construction to others described herein, including a balloon 980 near a distal end thereof (shown in a collapsed or deflated condition in FIGS. 8A-B) and a prosthetic heart valve PHV may be configured to be crimped over the balloon 980 during delivery. The balloon catheter 910 may extend to a distal tip 955 and may include a shaft 951 fixedly coupled to the distal tip 955. It should be understood that other interior components, such as a guidewire lumen or balloon inflation lumen, are omitted from FIGS. 8A-B for clarity. The shaft of the balloon catheter 910 may include a protective member 954 in the form of a highly compressible material that is flanked on either side by more traditional catheter materials. For example, in the illustrated embodiment of FIGS. 8A-B, the main portion of catheter shaft just, including for a short distance distal to the balloon 980, is formed of a traditional material, such as a braid-reinforced, reflowed shaft with a polymer (e.g., Pebax) jacket which is semi-rigid but allows for maneuverability of the balloon catheter 910 through the vasculature. The polymer jacket may have a Shore D durometer of between about 25D and about 72D, including about 40D, and may be formed of Pebax (as described above), or other materials including Pellethane, nylon, or polyethylene including high density polyethylene. The catheter shaft from the distal tip 955 to a distance slightly proximal to the distal tip 955 may be formed of the same material as the main portion of the catheter shaft. However, an intermediate section between the two above-described sections may be formed of a highly compressible material, an example of which is silicone or urethane having a Shore durometer of between about 20 and about 80 Shore A durometer. In some embodiments, instead of a material like silicone or urethane, a more traditional polymer material like Pebax may be used, but with a thin wall to provide the desired material properties. Rather than forming protective member 954 from a highly compressible material, in some embodiments (similar to that described below in connection with FIGS. 8D-E), the protective member 954 may be formed as a nitinol braid or laser cut material with a heat-set shape (e.g., set to take either the shape shown in FIG. 8A or shown in FIG. 8B), with force being applied to transition the protective member 954 away from the set shape. In some embodiments, instead of forming the intermediate section as a highly compressible material, it may be formed as a multi-layer material with a softer outer layer and a more rigid inner layer, so that as the inner layer is retracted, the outer layer expands. This intermediate section is preferably distal to the balloon 980 and the prosthetic heart valve PHV carried thereon. In use, the balloon catheter 910 may be passed through a patient's vasculature with the protective member 954 in the stowed condition in which the protective member has a diameter that is about equal to adjacent portions of the balloon catheter shaft and is smaller than the diameter of the deflated balloon 980 and prosthetic heart valve PHV crimped thereon. At the desired point during delivery, which may be just prior to reaching the aortic arch, or any other point when the balloon catheter 910 is inserted within the patient, the shaft 951 may be pulled proximally. By pulling the shaft 951 proximally, the distal tip 955 is also pulled proximally. Because of the high compressibility (and/or low Shore durometer) of the material forming the protective member 954, the protective member 954 will tend to expand outwardly into the deployed condition shown in FIG. 8B as the distal-most section of the shaft of the balloon catheter 910 retracts towards the main section of the shaft of the balloon catheter 910. Thus, pulling shaft 951 proximally activates the protective member 954, preferably so that the diameter of the protective member 954 is greater than the diameter of the deflated balloon 980 and the prosthetic heart valve PHV crimped thereon. With the protective member 954 in the activated or expanded condition, the balloon catheter 910 may be advanced, with the protective member 954 tending to ensure that the leading edge of the prosthetic heart valve PHV is not able to unintentionally contact tissue which could result in the position and/or orientation of the prosthetic heart valve PHV shifting undesirably relative to the balloon 980 and/or balloon catheter 910. Prior to deploying the prosthetic heart valve PHV by inflating the balloon 980, if desired the protective member 954 may be transitioned back to the stowed condition by advancing the shaft 951 distally. Thus, it is preferable that the shaft 951 is capable of transmitting both tension and distally directed axial forces. However, in other embodiments, the shaft 951 may be capable of only transmitting tension, and the protective member 954 may tend to revert back to the stowed condition of FIG. 8A when tension on the shaft 951 is released. It should also be understood that, although only a single protective member 954 is illustrated in FIGS. 8A-B, in another embodiment, a second protective member similar or identical to that shown may be positioned just proximal to the balloon 980, such that the balloon 980 (and prosthetic heart valve PHV mounted thereon) are flanked on both distally and proximally by an expanded protective member when the protective members are deployed. This second proximal protective member may help avoid contact of the trailing end of the prosthetic heart valve PHV with tissue, as well as helping center the prosthetic heart valve PHV within the vasculature and helping with tracking around the aortic arch. Further, as explained above, the protective members described herein may also be useful in providing protection to the prosthetic heart valve PHV when either inserting the prosthetic heart valve PHV into the patient, or even withdrawing a non-deployed prosthetic heart valve PHV from the patient through the vasculature and the insertion sheath if a procedure is being aborted.

FIG. 8C illustrates a highly schematic view of a distal end portion of a balloon catheter 910′ generally similar in function and structure to balloon catheter 910. As shown in FIG. 8C, balloon catheter 910′ includes a nosecone 955′ at a distalmost end. The main shaft 956′ of balloon catheter 910′ is illustrated without a balloon mounted to the shaft, but it should be understood that an inflatable balloon similar to other embodiments described herein would be mounted on the main shaft 956′ a short distance proximal of the nosecone 955′. An interior shaft 951′ is also shown, with a distal end of the shaft 951′ fixedly mounted to an interior portion of the nosecone 955′. For example, the shaft 951′ may be hollow to allow for a guidewire to pass therethrough, and a distal end of the shaft 951′ may be directly coupled to an interior of the nosecone 955′, or a coupling member such as a ring may be used to facilitate that coupling. In the illustrated embodiment, the main shaft 956′ is formed of a traditional catheter material such as those described above in connection with balloon catheter 910. However, at least the portion of nosecone 955′ proximal to the connection of shaft 951′, and distal to the portion of main shaft 956′ where the balloon would be mounted, is formed of a highly compressible material similar to those described above in connection with protective member 954. In one embodiment, only the portion of the nosecone 955′ proximal to the connection of shaft 951′ is formed of the highly compressible material so that the nosecone is formed of different materials along the length of the nosecone. In some embodiments, the entire nosecone 955′ may be formed of the same material, but the proximal portion of the nosecone may include features (such as slits in the material) to allow for that proximal portion to expand to form the protective member 954′. With this configuration, similar to balloon catheter 910, when shaft 956′ is pulled proximally, a portion of the nosecone 955′ expands radially outwardly to form a protective member 954′. As with the embodiment of FIGS. 8A-B, when in the deployed condition shown in FIG. 8C, the protective member 954′ has a diameter that is larger than the diameter of the crimped prosthetic heart valve PHV mounted on the deflated balloon coupled to main shaft 956′. In the illustrated embodiment, the height H1 of the nosecone 955′ may be about twice the height H2 between the point of coupling of the shaft 951′ to the nosecone 955′ and the distal end of the nosecone 955′. This particular relationship may not be critical, but it should be understood that the height H2 represents the portion of the nosecone 955′ that may remain unchanged, and that height H2 is preferably large enough to allow the nosecone 955′ to still perform its role as the leading atraumatic tip of the delivery device. The use of balloon catheter 910′ may be substantially similar or identical to the use of balloon catheter 910 described above and is thus not repeated again here.

FIGS. 8D-E illustrate a balloon catheter 910″ with a protective member 954″ in stowed and deployed conditions, respectively. Balloon catheter 910″ may be identical to balloon catheter 910, but for the protective member 954″, so the other components are not described again. Rather than the protective member 954″ being formed from a highly compressible (e.g., low durometer) material that expands outwardly under compression, as with protective member 954, protective member 954″ may be formed with a shape-memory material. For example, protective member 954″ may be formed as a general stent-like structure or mesh or latticework of a shape memory material, such as a nickel titanium alloy like nitinol. It should be understood that a coating or other covering may be provided on the mesh or lattice structure so that the inside of the balloon catheter 910″ is sealed from the outside across the protective member 954″. However, the covering is preferably formed of a soft or compressible material so that the material does not significantly hinder the protective member 954″ from transitioning between the stowed condition shown in FIG. 8D and the deployed or actuated condition shown in FIG. 8E. Referring to FIG. 8D, protective member 954″ is illustrated with zig-zag lines, but it should be understood that this section of the catheter 910″ may be generally cylindrical with in this stowed condition, with the same or similar outer diameter as the portions of the catheter 910″ just proximal and just distal to the protective member 954″. The protective member 954″ may include a braided mesh or laser cut tube of nitinol, as two examples, which may be shape set (e.g., via heat setting) to form a generally conical or umbrella shape in the absence of applied forces. As shown in FIG. 8E, in this set shape, the open proximal end of the conical or umbrella shape may extend radially beyond the outer extent of the crimped prosthetic heart valve PHV and may optionally axially cover a portion of the prosthetic heart valve PHV, for example overlying the leading edge of the prosthetic heart valve PHV. In use, the protective member may be in the stowed condition of FIG. 8D upon entry in the patient, for example so that a generally smooth surface is provided at the protective member 954″, and so that a generally smooth transition is provided where the two ends of the protective member 954″ join the more typically fashioned portions of the shaft of the catheter 910″. If the protective member 954″ is shape-set to have a generally conical or umbrella shape shown in FIG. 8E, the shaft 951 may be used to maintain the distal tip 955 in the position shown in FIG. 8D, in order to resist the protective member 954″ from changing shape. At the desired point during delivery, for example just prior to traversing the aortic arch, the shaft 951 may be pulled proximally, which, along with the protective member 954″ “wanting” to return to its set shape, causes the protective member 954″ to form a protective cap 954″. As with other embodiments described herein, the leading edge of the prosthetic heart valve PHV is thus protected from contacting tissue, which may otherwise cause an undesirable shift in the position and/or orientation of the prosthetic heart valve PHV relative to the balloon 980 and/or balloon catheter 910″. Prior to deploying the prosthetic heart valve PHV, the shaft 951 may be advanced to transition the protective member 954″ back toward the stowed condition to reduce potential interference with the prosthetic heart valve PHV as it is deployed. It should be understood that, even if protective member 954″ is shape-set to have the conical or umbrella shape shown in FIG. 8E, the biasing force may not be strong enough to transition from the shape shown in FIG. 8D to the shape shown in FIG. 8E without assistance, e.g., from pulling shaft 951. In other words, even though the protective member 954″ may be shape-set to a conical or umbrella shape, it may not spontaneously take that shape during use of the balloon catheter 910″ without additional force being applied to overcome other forces at play, such as the connection points to the more rigid portions of the main shaft of the balloon catheter 910″.

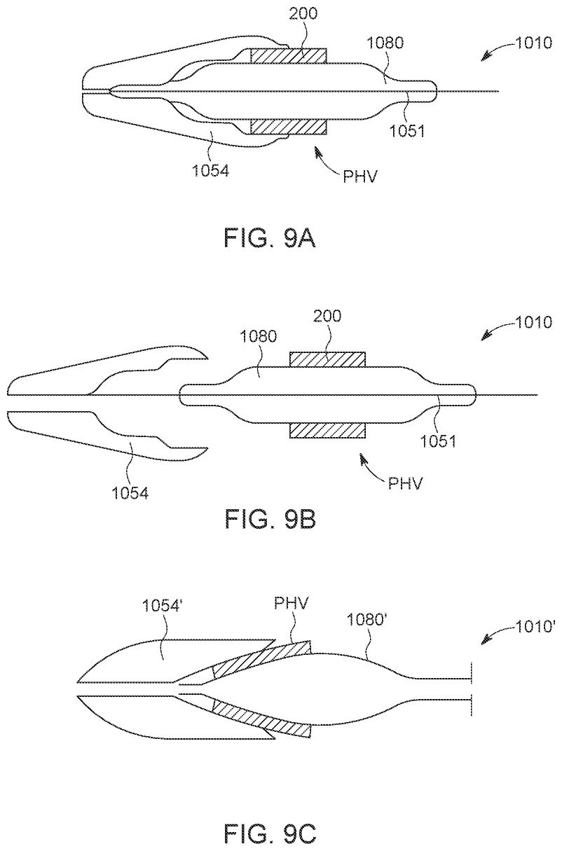

FIGS. 9A-B are schematic views of a balloon catheter 1010 according to another aspect of the disclosure. Similar to FIG. 5C, a number of components of balloon catheter 1010 are omitted from the view of FIGS. 9A-B, including the shaft on which balloon 1080 is mounted, and proximal segments of the balloon catheter 1010, including a control handle. FIG. 9A illustrates the nosecone 1054 of the balloon catheter 1010 in a stowed condition for delivery of the prosthetic heart valve PHV. The nosecone 1054 may be formed as a generally conical or bullet-shaped member, preferably with an atraumatic tip and a gentle taper from the largest proximal-most section to the narrowed distal-most section. The nosecone 1054 may be formed as a single integral member, despite the cross-section views of FIGS. 9A-B suggesting the nosecone 1054 may be two separate, unconnected pieces. As best shown in FIG. 9B, the nosecone 1054 may form an interior cavity that is generally complementary to the shape(s) of the deflated balloon 1080 and the prosthetic heart valve PHV crimped thereon. For example, the distal-most section (to the left in the views of FIGS. 9A-B) may be substantially solid but for a central lumen extending therethrough, the lumen sized and shaped to accept a guidewire (not shown) therethrough. An axially central portion of the nosecone 1054 may have an interior cavity that is generally shaped to receive the distal end portion of the deflated balloon, as best shown in FIG. 9A. A proximal-most section of the nosecone 1054 may include an even larger interior cavity than the central portion, that cavity being sized so that the proximal-most section of the nosecone 1054 receives the leading end of the prosthetic heart valve PHV which is mounted on the deflated balloon 1080.

Balloon catheter 1010 may include an interior shaft 1051 coupled to the nosecone 1054, similar to embodiments described above. The shaft 1051 may be a solid member or a member with a lumen, for example sized and shaped to accept a guidewire (not illustrated) therethrough. In either case, the shaft 1051 is preferably capable of transmitting pushing and pulling forces to the nosecone 1054. In use, prior to advancing the balloon catheter 1010 into the patient, the nosecone 1054 is pulled proximally (e.g., by pulling shaft 1051 proximally) after the prosthetic heart valve PHV is crimped onto the deflated balloon 1080. Once the distal end of the balloon 1080 is received within an interior cavity of the nosecone 1054, and the proximal end of the nosecone 1054 overlies the leading edge of the prosthetic heart valve PHV, the balloon catheter 1010 may be advanced into and through the patient's vasculature. As long as the proximal end of the nosecone 1054 overlies the leading edge of the prosthetic heart valve PHV, the leading edge of the prosthetic heart valve PHV is protected against contacting tissue unintentionally and shifting the position of the prosthetic heart valve PHV. When the prosthetic heart valve PHV is located within or adjacent the intended target site (e.g., the native aortic valve), the nosecone 1054 may be advanced distally, for example by distally sliding the shaft 1051. Once the proximal end of the nosecone 1054 is clear of the prosthetic heart valve PHV (and preferably also clear of the balloon 1080), the balloon 1080 may be inflated to deploy the prosthetic heart valve PHV without interference from the nosecone 1054. After deployment, the balloon 1080 may be deflated by withdrawing fluid from the balloon 1080, and the nosecone 1054 may be pulled proximally to again receive the distal end of the balloon 1080 before withdrawing the balloon catheter 1010 from the patient.

One potential drawback of the configuration of balloon catheter 1010 shown in FIGS. 9A-B is that the profile of the assembly may be large relative to a similar device with a nosecone that does not overlie the prosthetic heart valve PHV. In other words, the thickness of the nosecone 1054 where it covers the prosthetic heart valve PHV adds to the overall diameter of the assembly during delivery, and it is typically desirable to minimize the diameter of the assembly during delivery, where possible. Thus, FIG. 9C shows an alternate configuration of balloon catheter 1010′ which is similar to that of FIGS. 9A-B in most respects. The main difference is that the prosthetic heart valve PHV is crimped over the balloon 1080′ with a tapered configuration, where the leading edge of the prosthetic heart valve PHV is positioned closer to the longitudinal center of the balloon catheter 1010′ than is the trailing end of the prosthetic heart valve PHV. The nosecone 1054′ may be similar to nosecone 1054, with the main difference being the internal cavity of the nosecone 1054′ may have a complementarily tapered profile to receive the leading edge of the prosthetic heart valve PHV therein. With this configuration, the largest outer diameter of the nosecone 1054′ may be slightly smaller than the largest outer diameter of nosecone 1054, allowing for a smaller profile during delivery. The balloon catheter 1010′ otherwise operates identically to balloon catheter 1010. It should be understood that, even though the prosthetic heart valve PHV is crimped with a tapered profile onto balloon 1080′, upon expansion of the balloon 1080′ during deployment, the balloon 1080′ and prosthetic heart valve PHV may take generally cylindrical shapes.

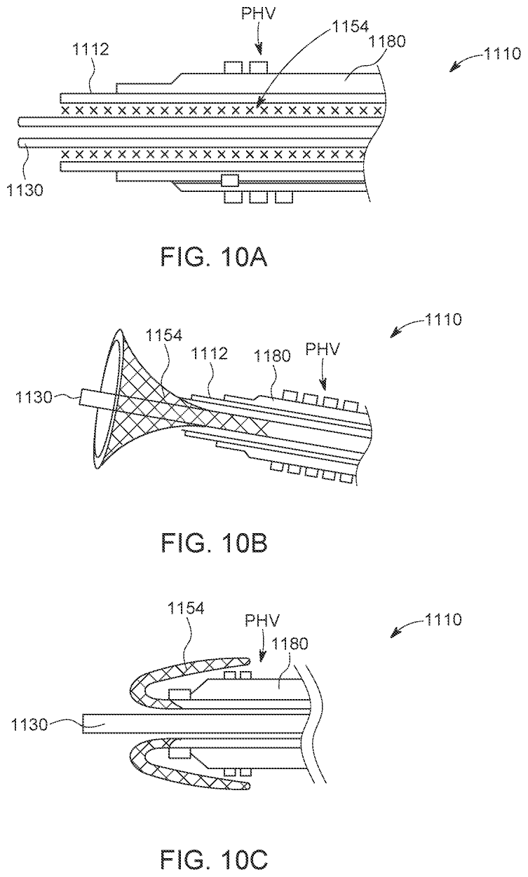

FIGS. 10A-B are schematic illustrations of a distal end portion of a balloon catheter 1110 according to another aspect of the disclosure. As with other embodiments described herein, balloon catheter 1110 may include a balloon 1180 mounted to a shaft 1112 of the balloon catheter 1110, and a prosthetic heart valve PHV may be crimped onto the deflated balloon 1180 for delivery. A guidewire lumen 1130 may pass through a center portion of the balloon catheter 1110 so that the balloon catheter 1110 may ride over a guidewire (not illustrated) previously inserted into the patient. The material forming the guidewire lumen 1130 may be any suitable material, including for example braided or non-braided polyimide, or braided or non-braided high-density polyethylene (“HDPE”). Although not shown, balloon catheter 1110 may still include an atraumatic distal tip or nosecone distal to the protective member 1154, which is described in greater detail below.

A protective member 1154 may be positioned inside the shaft 1112 of the balloon catheter 1110, and outside the guidewire lumen 1130, near or at the distal end of the shaft 1112. In the illustrated embodiment, the protective member 1154 may be formed as a braided structure that is generally cylindrical while stowed within the balloon catheter 1110. The material forming the braided structure of the protective member 1154 may be a polymer or a shape-memory alloy such as nickel titanium (including nitinol). If formed of a shape-memory material, the protective member 1154 may be shape set as described below, but it should be understood that protective member 1154 may function as intended without having a particular set shape. Although not shown in FIGS. 10A-C, the protective member 1154 may extend (or a connector member may extend) proximally, for example to a handle of the delivery device incorporating the balloon catheter 1110, so that the protective member 1154 may be translated distally or proximally relative to the shaft 1112 of the balloon catheter 1110.

During delivery of prosthetic heart valve PHV, the protective member 1154 may be in the stowed condition with the protective member 1154 fully inside of the shaft 1112 of the balloon catheter 1110. When it is desired to activate the protective functionality of protective member 1154 (e.g., just before traversing the aortic arch), the user may slide the protective member 1154 distally. As shown in FIG. 10B, as the distal end of the protective member 1154 clears the constraints of the shaft 1112 of the balloon catheter 1110, allowing the distal end of the protective member 1154 to expand. In some embodiments, the protective member 1154 may be shape-set to expand to the configuration shown in FIG. 10B. In some embodiments, the mere release of constraints on the protective member 1154 may cause or allow the protective member 1154 to begin to expand.

The distal-most end of the protective member 1154 may maintain the generally conical or frustoconical shape shown in FIG. 10B as the balloon catheter 1110 is advanced after activation of the protective member 1154. In the event that the distal end of the protective member 1154 comes into contact with tissue during delivery, the distal end of the protective member 1154 may be forced to flip backwards, as shown in FIG. 10C, so that the protective member 1154 covers the leading edge of the prosthetic heart valve PHV. When the leading edge of the prosthetic heart valve PHV is covered by the protective member 1154, the prosthetic heart valve PHV will be protected from directly contacting tissue and having its position or orientation undesirable changed. In some embodiments, if the protective member 1154 is formed of a shape-memory material, the protective member 1154 may be shape-set to the condition shown in FIG. 10C. When it is time to deploy the prosthetic heart valve PHV, whether the protective member 1154 is in the condition shown in FIG. 10B or 10C, the protective member 1154 may be retracted by pulling it proximally, forcing the protective member 1154 to slide back into the space between the shaft 1112 and the guidewire lumen 1130 of the balloon catheter 1110. Then, the prosthetic heart valve PHV may be deployed via expansion of balloon 1180 without interference from the protective member 1154.

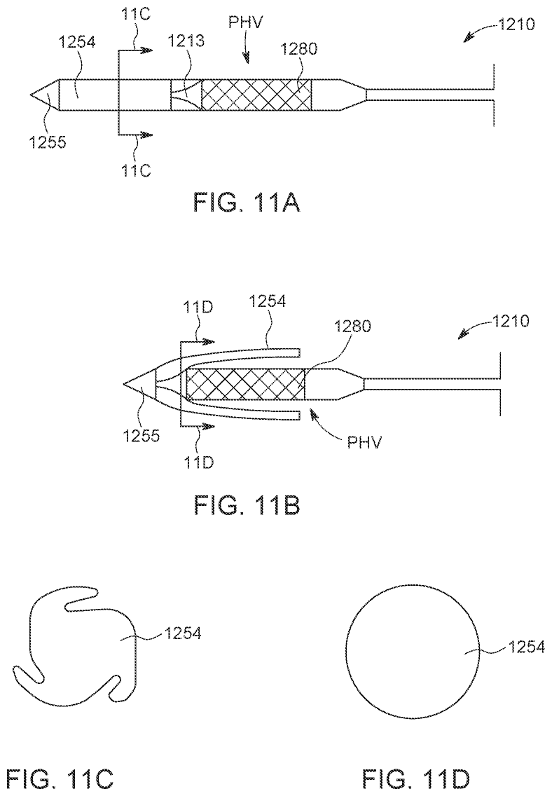

FIGS. 11A-B are schematic illustrations of a distal end portion of a balloon catheter 1210 according to another aspect of the disclosure. As with other embodiments described herein, balloon catheter 1210 may include a balloon 1280 mounted to a shaft of the balloon catheter 1210, and a prosthetic heart valve PHV may be crimped onto the deflated balloon 1280 for delivery. A guidewire lumen (not shown) may pass through a center portion of the balloon catheter 1210 so that the balloon catheter 1110 may ride over a guidewire (not illustrated) previously inserted into the patient. An atraumatic tip 1255 may be provided at the distal-most end of the balloon catheter 1210.

A protective member 1254 in the form of a capsule may be provided proximal to (and coupled to) the distal tip 1255. The capsule 1254 and/or tip 1255 may be coupled to an internal shaft (not illustrated) that may be translated relative to the balloon 1280 in a similar manner as described above for other shafts described herein, such as shafts 951, 1051. In the extended or stowed condition, as shown in FIG. 11A, the capsule 1254 may be generally cylindrical. However, as shown in FIG. 11C, the general cylindrical shape may include one or more folded areas to reduce the profile of the capsule 1254 when in the stowed condition. When in this extended or stowed condition, the balloon catheter 1210 may be passed through an introducer and into the patient's vasculature.

At any point after the balloon catheter 1210 has entered the patient's vasculature, the capsule 1254 may be transitioned to the deployed condition to protect the prosthetic heart valve PHV. In order to complete this transition, the inner shaft coupled to the distal tip 1255 and/or capsule 1254 may be retracted. As the capsule 1254 is retracted relative to the prosthetic heart valve PHV, the capsule 1254 will slide along ramped portion 1213, forcing the capsule to unfold and increase in internal diameter as it overlies the prosthetic heart valve PHV. As shown in FIG. 11D, when unfolded, the capsule 1254 may be substantially cylindrical without (or with minor) folds such that it is large enough to cover the prosthetic heart valve PHV. This is compared to the extended or stowed condition, in which the outer diameter of the capsule 1254 is about equal to the outer diameter of the deflated balloon 1280 and/or crimped prosthetic heart valve PHV, due to the folds of the capsule 1254. In one embodiment, the capsule 1254 may be formed with a low friction liner such as HDPE or PTFE, along with a reflowed polymer jacket like Pebax. A hydrophilic coating may also be provided to the outside of capsule 1254 for even smoother navigation. Similar hydrophilic coatings may be provided to other embodiments described herein for similar purposes. The use of balloon catheter 1210 may be substantially similar to other embodiments described herein and is thus not described again in detail here.

According to one aspect of the disclosure, a prosthetic heart valve system comprises:
a balloon expandable prosthetic heart valve; and
a delivery catheter having a balloon on a distal end portion of the delivery catheter, wherein in a delivery condition of the system and in an activated condition of the system, the prosthetic heart valve is mounted over the balloon so that a leading edge of the prosthetic heart valve points toward a distal end of the delivery catheter and so that the prosthetic heart valve has an outer radial extent,
wherein the delivery catheter includes a protective member operably coupled to the delivery catheter, and in the delivery condition of the system, the protective member does not extend radially beyond the outer radial extent of the prosthetic heart valve, and in the activated condition of the system, the protective member does extend radially beyond the outer radial extent of the prosthetic heart valve; and/or
the delivery catheter includes a plurality of peripheral lumens extending through a sidewall thereof, and the protective member includes a plurality of protective wires, each protective wire extending through a corresponding one of the peripheral lumens; and/or
each of the plurality of protective wires is formed of a shape memory material; and/or
each of the plurality of protective wires had a distal free end, the distal free ends being positioned within the corresponding ones of the peripheral lumens in the delivery condition of the system, the distal free ends extending beyond the peripheral lumens and curling proximally in the activated condition of the system; and/or
each of the plurality of protective wires has a distal end, the distal ends of the protective wires each being fixed to respective point of connection to an outer surface of the delivery catheter such that a distal portion of each of the plurality of protective wires exits the corresponding one of the peripheral lumens and turns proximally to the point of connection; and/or
the protective member is a protective wire having a main shaft and a plurality of distal wire end portions extending from the main shaft, the distal wire portions forming an umbrella shape in the absence of applied forces; and/or
a covering material coupled to the distal wire portions of the protective wire; and/or
in the activated condition of the system, the distal wire portions of the protective wire overlie the leading edge of the prosthetic heart valve; and/or
the protective member includes a shaft and an inflatable member at the distal end of the shaft, the inflatable member having a hemispherical or bullet shape when inflated, the inflatable member being inflate in the activated condition of the system; and/or
the shaft of the protective member is translatable relative to the delivery catheter, and the inflatable member is received within the delivery catheter when the inflatable member is deflated in the delivery condition; and/or
the protective member is directly coupled to the delivery catheter distal to the balloon, the protective member being an inflatable member that is deflated in the delivery condition of the system and inflated in the activated condition of the system; and/or
the protective member is a portion of the delivery catheter being positioned distal to the balloon, the protective member having a compressibility that is greater than a compressibility of other portions of the delivery catheter adjacent the protective member; and/or
a shaft coupled to a distal tip of the delivery catheter, wherein retracting the shaft proximally pulls the distal tip proximally to transition the prosthetic heart valve system from the delivery condition to the activated condition; and/or
the protective member is a portion of the delivery catheter being positioned distal to the balloon, the protective member including a braided mesh formed of shape memory material; and/or
in the delivery condition of the system, the protective member is generally cylindrical, and in the activated condition of the system, the braided mesh forms a conical or umbrella shape that covers the leading edge of the prosthetic heart valve; and/or
the protective member is a generally cylindrical braided mesh housed within the delivery catheter radially inward of the balloon in the delivery condition of the system; and/or
the protective member is translatable relative to the balloon, a distal end portion of the protective member extending beyond a distal end of the delivery catheter in the activated condition of the system; and/or
the protective member is a capsule positioned distal to the balloon when the system is in the delivery condition, the protective member including a plurality of folds in the delivery condition of the system; and/or
the capsule overlies the balloon and the prosthetic heart valve in the activated condition of the system, the protective member excluding the plurality of folds in the activated condition of the system.

According to another aspect of the disclosure, a prosthetic heart valve system comprises:
a balloon expandable prosthetic heart valve; and
a delivery catheter having a balloon on a distal end portion of the delivery catheter, wherein in a delivery condition of the system and in an activated condition of the system, the prosthetic heart valve is mounted over the balloon so that a leading edge of the prosthetic heart valve points toward a distal end of the delivery catheter,
wherein the delivery catheter includes a nosecone coupled to a shaft that extends though the delivery catheter, the shaft being translatable relative to the balloon to translate the nosecone relative to the balloon,
wherein in a delivery condition of the system, the nosecone is positioned at a first axial location relative to the balloon so that a portion of the balloon and the leading edge of the prosthetic heart valve are covered by a proximal portion of the nosecone, and
wherein in a deployment condition of the system, the nosecone is positioned at a second axial location relative to the balloon distal to the first axial location, so that the proximal portion of the nosecone does not overlie the leading edge of the prosthetic heart valve.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve system, comprising:
a balloon expandable prosthetic heart valve; and
a delivery catheter having a balloon on a distal end portion of the delivery catheter, wherein in a delivery condition of the system and in an activated condition of the system, the prosthetic heart valve is mounted over the balloon so that a leading edge of the prosthetic heart valve points toward a distal end of the delivery catheter and so that the prosthetic heart valve has an outer radial extent,
wherein the delivery catheter includes a protective member operably coupled to the delivery catheter, and in the delivery condition of the system, the protective member does not extend radially beyond the outer radial extent of the prosthetic heart valve, and in the activated condition of the system, the protective member does extend radially beyond the outer radial extent of the prosthetic heart valve.

2. The prosthetic heart valve system of claim 1, wherein the delivery catheter includes a plurality of peripheral lumens extending through a sidewall thereof, and the protective member includes a plurality of protective wires, each protective wire extending through a corresponding one of the peripheral lumens.

3. The prosthetic heart valve system of claim 2, wherein each of the plurality of protective wires is formed of a shape memory material.

4. The prosthetic heart valve system of claim 3, wherein each of the plurality of protective wires had a distal free end, the distal free ends being positioned within the corresponding ones of the peripheral lumens in the delivery condition of the system, the distal free ends extending beyond the peripheral lumens and curling proximally in the activated condition of the system.

5. The prosthetic heart valve system of claim 2, wherein each of the plurality of protective wires has a distal end, the distal ends of the protective wires each being fixed to respective point of connection to an outer surface of the delivery catheter such that a distal portion of each of the plurality of protective wires exits the corresponding one of the peripheral lumens and turns proximally to the point of connection.

6. The prosthetic heart valve system of claim 1, wherein the protective member is a protective wire having a main shaft and a plurality of distal wire end portions extending from the main shaft, the distal wire portions forming an umbrella shape in the absence of applied forces.

7. The prosthetic heart valve system of claim 6, further comprising a covering material coupled to the distal wire portions of the protective wire.

8. The prosthetic heart valve system of claim 5, wherein in the activated condition of the system, the distal wire portions of the protective wire overlie the leading edge of the prosthetic heart valve.

9. The prosthetic heart valve system of claim 1, wherein the protective member includes a shaft and an inflatable member at the distal end of the shaft, the inflatable member having a hemispherical or bullet shape when inflated, the inflatable member being inflate in the activated condition of the system.

10. The prosthetic heart valve system of claim 9, wherein the shaft of the protective member is translatable relative to the delivery catheter, and the inflatable member is received within the delivery catheter when the inflatable member is deflated in the delivery condition.

11. The prosthetic heart valve system of claim 1, wherein the protective member is directly coupled to the delivery catheter distal to the balloon, the protective member being an inflatable member that is deflated in the delivery condition of the system and inflated in the activated condition of the system.

12. The prosthetic heart valve system of claim 1, wherein the protective member is a portion of the delivery catheter being positioned distal to the balloon, the protective member having a compressibility that is greater than a compressibility of other portions of the delivery catheter adjacent the protective member.

13. The prosthetic heart valve system of claim 12, further comprising a shaft coupled to a distal tip of the delivery catheter, wherein retracting the shaft proximally pulls the distal tip proximally to transition the prosthetic heart valve system from the delivery condition to the activated condition.

14. The prosthetic heart valve system of claim 1, wherein the protective member is a portion of the delivery catheter being positioned distal to the balloon, the protective member including a braided mesh formed of shape memory material.

15. The prosthetic heart valve system of claim 14, wherein in the delivery condition of the system, the protective member is generally cylindrical, and in the activated condition of the system, the braided mesh forms a conical or umbrella shape that covers the leading edge of the prosthetic heart valve.

16. The prosthetic heart valve system of claim 1, wherein the protective member is a generally cylindrical braided mesh housed within the delivery catheter radially inward of the balloon in the delivery condition of the system.

17. The prosthetic heart valve system of claim 16, wherein the protective member is translatable relative to the balloon, a distal end portion of the protective member extending beyond a distal end of the delivery catheter in the activated condition of the system.

18. The prosthetic heart valve system of claim 1, wherein the protective member is a capsule positioned distal to the balloon when the system is in the delivery condition, the protective member including a plurality of folds in the delivery condition of the system.

19. The prosthetic heart valve system of claim 18, wherein the capsule overlies the balloon and the prosthetic heart valve in the activated condition of the system, the protective member excluding the plurality of folds in the activated condition of the system.

20. A prosthetic heart valve system, comprising:
a balloon expandable prosthetic heart valve; and
a delivery catheter having a balloon on a distal end portion of the delivery catheter, wherein in a delivery condition of the system and in an activated condition of the system, the prosthetic heart valve is mounted over the balloon so that a leading edge of the prosthetic heart valve points toward a distal end of the delivery catheter,
wherein the delivery catheter includes a nosecone coupled to a shaft that extends though the delivery catheter, the shaft being translatable relative to the balloon to translate the nosecone relative to the balloon,
wherein in a delivery condition of the system, the nosecone is positioned at a first axial location relative to the balloon so that a portion of the balloon and the leading edge of the prosthetic heart valve are covered by a proximal portion of the nosecone, and
wherein in a deployment condition of the system, the nosecone is positioned at a second axial location relative to the balloon distal to the first axial location, so that the proximal portion of the nosecone does not overlie the leading edge of the prosthetic heart valve.
